(19) United States
(12) Patent Application Publication
(10) Pub. No.: US 2021/0198312 A1
(43) Pub. Date: Jul. 1, 2021

(54) GROWTH FACTOR CONCENTRATE AND METHOD OF MANUFACTURE THEREOF

(71) Applicant: Helena Lovick, N. Great Falls, MT (US)

(72) Inventor: Helena Lovick, N. Great Falls, MT (US)

(21) Appl. No.: 17/138,261

(22) Filed: Dec. 30, 2020

Related U.S. Application Data
(60) Provisional application No. 62/955,886, filed on Dec. 31, 2019.

(51) Int. Cl. C07K 1/36; C07K 14/51; C07K 14/495; C07K 14/475; C07K 14/50; A61L 27/22; A61L 27/36

(52) U.S. Cl. CPC C07K 1/36; C07K 14/51; C07K 14/495; A61K 38/00; C07K 14/50; A61L 27/227; A61L 27/3608; C07K 14/475

(57) ABSTRACT

The present invention relates to a growth factor concentrate and methods of making and using the same.

### CROSS REFERENCE TO RELATED APPLICATIONS

This application claims priority and the benefit under 35 U.S.C. § 119(e) to U.S. Provisional Patent Application Ser. No. 62/955,886, filed Dec. 31, 2019. This reference is incorporated herein in its entirety by reference.

### FIELD OF THE INVENTION

The present embodiments generally relate to bone marrow purification to produce a growth factor concentrate and method of manufacture of same.

### BACKGROUND

Bone marrow is found within the interior of bones and its composition may range from almost exclusively fatty tissue to highly cellularized red marrow with a high degree of heterogeneity based on the individual and the marrow location. It is well known that bone marrow has a wide range of therapeutic properties. Bone marrow has been transplanted in humans to treat various health conditions and diseases for over sixty years. Because bone marrow aspirate (BMA) contains a variety of platelets, blood cells, and stem cells, methods have been developed to collect, process, and transplant bone marrow.

U.S. Pat. No. 5,486,359 entitled “Human mesenchymal stem cells” (incorporated by reference in its entirety) discloses an isolated, homogeneous population of human mesenchymal stem cells (hMSCs) which can differentiate into cells of more than one connective tissue type. In one example, hMSCs were isolated from bone marrow aspirate via dispersion into a described aqueous medium and centrifuged to pellet the cells. The cell pellet was further purified via a Percoll density gradient medium.

U.S. Pat. No. 7,326,571 entitled “Decellularized bone marrow extracellular matrix” (incorporated by reference in its entirety) discloses a biocompatible material comprising decellularized bone marrow extracellular matrix. In one example, the disclosed bone marrow extracellular matrix is physically collected from bone samples, filtered, centrifuged in saline to remove fat. After fat removal, a non-ionic detergent is added to solubilize the cell membranes and fat. The sample is then rinsed with saline and filtered to remove additional extracellular matrix. The remaining sample is treated with DNase and RNase, then rinsed with saline to remove the remaining extracellular matrix.

U.S. Pat. No. 8,328,876 entitled “Bone matrix compositions and methods” (incorporated by reference in its entirety) discloses a bone matrix that has been exposed to collagenase to produce a modified bone matrix. The level of at least one biological activity of the disclosed bone matrix is increased relative to its level in an unmodified control bone matrix, wherein the biological activity is selected from the group consisting of osteoinductive activity, osteogenic activity, and chondrogenic activity.

U.S. Pat. No. 9,687,511 entitled “Acellular biologic composition and method of manufacture” (incorporated by reference in its entirety) discloses a biological composition with a mixture of material derived from bone marrow. The marrow processing separates the cellular components via filtration and density gradient centrifugation. The non-cellular composition is suspended in a cryoprotectant and frozen for storage.

U.S. Pat. No. 5,770,705 entitled “Method for recovering proteins from plasma using insoluble, water absorbing material” (incorporated by reference in its entirety) discloses a process for producing purified proteins from plasma by cold precipitation. The process includes the steps of contacting plasma with a water-absorbing, chromatographic gel, removing the gel from the plasma, chilling the plasma to precipitate proteins, and separating the protein precipitate from the liquid plasma.

### SUMMARY OF THE INVENTION

The disclosed invention pertains to an article comprised of a growth factor concentrate derived from bone marrow processed by a novel method. In some embodiments, the growth factor concentrate can be produced by a sequential centrifugation, acidification and neutralization process. In the course of the method of generating the growth factor concentrate, judicious selection of the acid type and concentration provides optimal growth factor concentration while demineralizing and decellularizing the bone marrow component. After treatment with acid, the bone marrow is further purified into the growth factor concentrate of the present invention via the addition of a basic agent. The basic agent is selected to neutralize the acidified material to a pH of greater than 6 but less than 9. In some embodiments, the basic agent serves to precipitate additional growth factors from the liquid portion of the composition. Other additives can be used during manufacture of the growth factor concentrate such as stabilizing buffer salts and preservatives. The growth factor concentrate derived from bone marrow has greater than about 1,000 picograms of at least one growth factor per gram of the isolated concentrate in the absence of other additives. In some embodiments the growth factor concentrate derived from bone marrow has as much as 100,000 nanograms of at least one growth factor per gram of the isolated concentrate in the absence of other additives. In some embodiments, the growth factor concentrate derived from bone marrow can be about 1,000 pg of at least one growth factor per gram of the isolated concentrate, about 5,000 ng of at least one growth factor per gram of the isolated concentrate, about 10,000 ng of at least one growth factor per gram of the isolated concentrate, about 20,000 ng of at least one growth factor per gram of the isolated concentrate, about 30,000 ng of at least one growth factor per gram of the isolated concentrate, about 40,000 ng of at least one growth factor per gram of the isolated concentrate, about 50,000 ng of at least one growth factor per gram of the isolated concentrate, about 60,000 ng of at least one growth factor per gram of the isolated concentrate, about 70,000 ng of at least one growth factor per gram of the isolated concentrate, about 80,000 ng of at least one growth factor per gram of the isolated concentrate, about 90,000 ng of at least one growth factor per gram of the isolated concentrate, about 100,000 ng of at least one growth factor per gram of the isolated concentrate, or a range between any two of the values, or a value or range within the largest range.

After isolation of the growth factor concentrate predominantly in the solid form (at room temperature) away from the processing liquids, in some embodiments, the growth factor concentrate may be combined with, but not limited to, antimicrobial agents, biodegradable polymers, biological tissues, preservatives, and combinations thereof. In some embodiments, the biological tissues can be bone. In some embodiments, the growth factor concentrate can be coated on the surface of an implant in the absence or presence of other materials such as combination with a biodegradable polymer for dip coating a bone tissue. A further aspect of the invention is a method of three-dimensional printing an osteoinductive implant containing the growth factor concentrate.

### BRIEF DESCRIPTION OF THE FIGURE

FIG. 1 illustrates a process flowchart for manufacturing the growth factor concentrate in an embodiment of the invention.

### DETAILED DESCRIPTION OF THE INVENTION

The present invention generally relates to bone marrow purification to produce a growth factor concentrate and methods of manufacturing the same.

“Allogeneic” or “allograft”, as used herein, refers to tissue derived from a non-identical donor of the same species, which may be a demineralized bone matrix (DBM).

“Antimicrobial agent” as used herein, broadly includes, but is not limited to, antibiotics, antimicrobials, antiseptics, and antifungals.

“Autogeneic” or “autograft”, as used herein, refers to tissue derived from and implanted into the same identical patient.

“Biocompatible”, as used herein, refers to the property of being biologically compatible with a living being by not causing harm.

“Biodegradable polymer” as used herein, is broadly defined as any polymer capable of being broken down by natural biological or environmental processes.

“Minimum inhibitory concentration” or “MIC”, as used herein, refers to the lowest concentration of a chemical which prevents visible growth of a microorganism.

“Osteoinductive”, as used herein, refers to the ability of a material to induce bone healing via recruitment of osteoprogenitor cells.

“Patient”, as used herein, refers to a living recipient of the biomaterial-based implants of the present invention.

“Room temperature”, as used herein, is defined as temperatures ranging from about 18 to about 27 degrees Celsius.

“Sieve”, as used herein, is defined as a tool used for separating coarser solids from finer solids and liquids.

“Xenogeneic” or “xenograft”, as used herein, is defined as tissue derived from a non-identical donor of a different species.

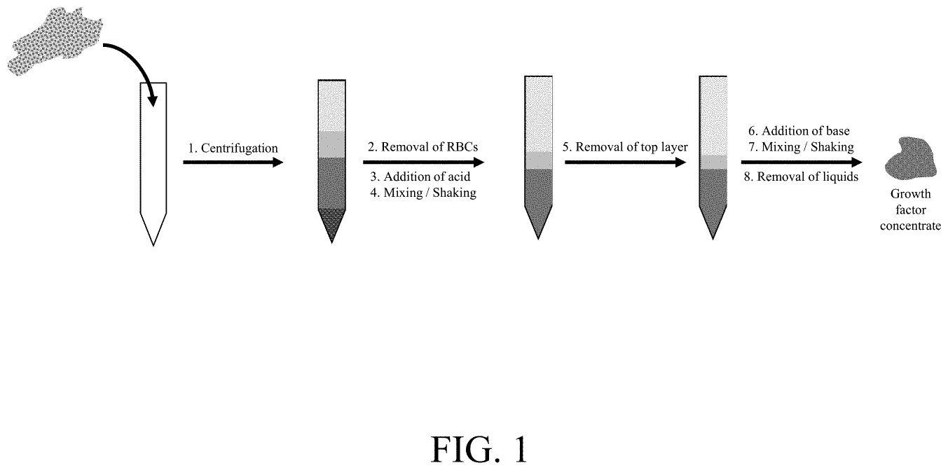

As illustrated in FIG. 1, one process for producing the growth factor concentrate can be broken into the following steps: (1) centrifugation, (2) removal of red blood cells (RBCs), (3) addition of acid, (4) mixing and/or shaking, (5) removal of the top liquid layer, (6) addition of base, (7) mixing and/or shaking, and (8) removal of the liquids to provide a growth factor concentrate.

Prior to Centrifugation (Prior to Step 1)

The collected bone marrow can be filtered through a sieve to remove bone material. The sieve may be sized from about 8 mm to about 1000 microns. In some embodiments, the sieve can be sized at about 8 mm, about 10 mm, about 20 mm, about 25 mm, about 30 mm, about 50 mm, about 75 mm, about 100 mm, about 150 mm, about 200 mm, about 250 mm, about 300 mm, about 350 mm, about 400 mm, about 450 mm, about 500 mm, about 550 mm, about 600 mm, about 650 mm, about 700 mm, about 750 mm, about 800 mm, about 850 mm, about 900 mm, about 950 mm or about 1000 mm or within any range defined between two of the foregoing numbers, or a value or range within the largest range. The particle size of the unprocessed bone marrow may be reduced through methods known in the art, such as grinding and milling, before passage through the sieve. In some embodiments, the bone material mixed within the unprocessed bone marrow may be retained throughout the purification process.

Step 1. Centrifugation

The centrifugation step may be performed at a range of standard operating conditions, for example at 2000 rpm for 14 minutes. In some embodiments, the centrifugation takes place at about 10,000 rpm to about 1,000 rpm, about 5,000 rpm to about 2,000 rpm. In some embodiments, the centrifugation can be about 1,000 rpm, about 2,000 rpm, about 3,000 rpm, about 5,000 rpm, about 7500 rpm, about 10,000 rpm, or within any range defined between two of the foregoing numbers, or a value or range within the largest range In other embodiments, the centrifugation takes place for about 2 minutes to about 20 minutes. In some embodiments, the time duration of the centrifugation can be about 2 minutes, about 5 minutes, about 10 minutes, about 15 minutes, about 20 minutes, or within any range defined between two of the foregoing numbers, or a value or range within the largest range. In further embodiments, the centrifugation may take place multiple times. In some embodiments, the centrifugation takes place at reduced temperatures. Reduced temperatures may range from about 2° C. to about 20° C. In some embodiments, the temperature can be about 2° C., about 5° C., about 8° C., about 10° C., about 15° C., or about 20° C. or within any range defined between two of the foregoing numbers, or a value or range within the largest range. The centrifuge rotor speed may be adjusted in order to provide the desired level of bone marrow separation into three or four distinct layers.

Step 2. Removal of RBCs

After centrifugation, at least three layers are present and correspond to a water-based layer, an oil-based layer and a solids layer. Each layer can be identified by its location within the tube. For example, the water-based layer can be identified by its higher density and subsequent partitioning into the bottom of the tube. Other layers may be present, including RBCs, solids and oils. In some embodiments, there may be more layers or each of the discussed layers can include additional layers. For example, the solids layer can include two layers corresponding to a lighter weight solid or a heavy weight solid. After centrifugation, the red blood cells (RBCs) may be removed from the bottom layer of the centrifuge container tube if present. If the removal of bone material is desired, the top layers above the bone and RBCs layer at the bottom of the container can be removed, by suction, decanting, or other similar technique and collected for further processing and the bottom layer, which can include dense bone pieces and RBCs can be discarded. In some embodiments, the layers can be further processed rather than discarding the layers.

Step 3. Addition of Acid

The concentration of the acid can be varied to result in a desired effect as would be understood by one skilled in the art. The acid can be any acid concentration capable of a pH of 3 or lower in an aqueous environment. The acid can be selected from the group of citric, hydrochloric, phosphoric, acetic, and mixtures thereof. The acid can be added to the bone marrow in a volume ratio of about 1:0.2 bone marrow to acid, to about 1:1 bone marrow to acid, to about 1:4 bone marrow to acid or within any range defined between two of the foregoing numbers, or a value or range within the largest range.

Step 4. Mixing/Shaking

After addition of the acid to the bone marrow, the mixture can be mixed thoroughly through various means known in the art, such as sonication, orbital shaking, vortexing, and combinations thereof. The duration of the mixing and shaking step can range from about 5 minutes to about 24 hours, from about 5 minutes to about 12 hours, from about 5 minutes to about 2 hours. In some embodiments, the duration can be about 5 minutes, about 10 minutes, about 15 minutes, about 30 minutes, about 45 minutes, about 1 hour, about 1.5 hours, about 2 hours, about 2.5 hours, about 3 hours, about 5 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours or within any range defined between two of the foregoing numbers, or a value or range within the largest range.

Step 5. Removal of Top Layer

After Step 4, “Mixing/Shaking” the processed material may be separated via sedimentation, centrifugation or other suitable technique. The acid treated material can be centrifugated to provide separation into two to three layers with the addition of solids settling to the bottom of the container. The solids can settle due to changes in its properties following the acid treatment. After separation into two or three layers, the top layer of the materials can be removed and discarded. The top layer may be removed by means known in the art such as use of a separatory funnel, decanting, pipetting, or other similar means of removal.

Step 6. Addition of Base

The remaining solids and liquids within the container are combined with a base until the pH is raised to 6 or greater. The base can be any concentration capable of raising the pH to 6 or greater while not overflowing the container space. The base can be selected from the group of hydroxide, silicate, phosphate, carbonate, acetate, citrate, or mixtures thereof. In some embodiments the base can be sodium hydroxide. During the addition of the base, the solution can be simultaneously mixed thoroughly and the temperature of the solution can be about 2° C. to about 25° C. In some embodiments, the pH after base addition can be between about 6 to about 10, about 6 to about 8, about 7. In some embodiments, the pH can be about 6, about 7, about 7.4, about 8, about 9, or about 10 or within any range defined between two of the foregoing numbers, or a value or range within the largest range.

Step 7. Mixing/Shaking

After the addition of the base to raise the pH of the solution to 6 or greater, the mixture can be thoroughly mixed as described in Step 4.

Step 8. Removal of Liquids

After Step 7, “Mixing/Shaking” the processed material can be separated via sedimentation, centrifugation or other suitable technique. The base treated material can be centrifugated to provide separation into at least two layers, in some embodiments two to three layers, with the addition of a solids layer at the bottom of the container. After separation into two or three layers, the liquid is removed from the solid materials. The liquid may be removed by means known in the art such as use of a filtration, decanting, pipetting, or other similar means of removal. After the bulk of the liquid is separated from the solids, the growth factor concentrate maybe further dried via evaporative drying, evaporative drying with added heat above room temperatures, lyophilization, or other suitable methods known in the art. In some embodiments, the material can be lyophilized to a residual moisture content of less than about 6%. The growth factor concentrate so derived from bone marrow has greater than about 1,000 picograms of at least one growth factor per gram of the isolated concentrate in the absence of other additives. In some embodiments, the growth factor concentrate can include at least one growth factor per gram of the isolate concentrate of between about 1,000 picograms and about 20,000 picograms. In some embodiments, the growth factor concentrate can be about 20,000 picograms, 10,000 picograms, about 1,000 picograms, about 100 picograms, or within any range defined between two of the foregoing numbers, or a value or range within the largest range. The growth factors contained within the growth factor concentrate include, but are not limited to, bone morphogenetic proteins (BMP) (which can include BMP-2, BMP-4, BMP-7, BMP-3, BMP-5, BMP-6, BMP-8, BMP-8a, BMP-8b, BMP-9, BMP-10, BMP-11, BMP-15, or combinations thereof), insulin-like growth factors (IGF) (which can include IGF-1, IGF-2, IGF-3, IGF-4, IGF-5, IGF-6, IGF-7, or combinations thereof), transforming growth factor beta, and fibroblast growth factors (which can include TGFB1, TGFB2, TGFB3, or combinations thereof). The layer where the growth factor can be found in the layered system can depend on the particular growth factor. For example, BMPs are generally located in the solids layer between the aqueous layer and the oily layer, or dissolved within the water based layer.

In some embodiments, biocompatible components can be added to the bone marrow during the processing. Suitable additives for inclusion during the purification process include preserving agents (e.g., cyropreservatives), antibiotics, and buffering acid and salt groups, and combinations thereof. The additives can be added at any stage of the purification process, for example during Step 6. Suitable antibiotics include, but are not limited to, silver sulfadiazine, chlorhexidine, gentamicin, tobramycin, vancomycin and combinations thereof. The mass ratio of the growth factor concentrate to the additives can be between about 10:1 and 0.01:1, about 1:1 to about 0.1 to 1, and ranges therein, based on the additive type. For additives such as antibiotics, buffering salt groups, the amount of additive to growth factor may be further reduced to provide the minimum inhibitory concentration for the selected antibiotic. For buffer salt groups the ratio of additive to growth factor concentrate may be about 0.01 to about 1, about 0.1 to about 1, about 1 to about 1, and ranges therein. Buffering acid and salt groups can include, but are not limited to, acetic acid/sodium acetate, potassium dihydrogen phosphate/disodium hydrogen phosphate, sodium bicarbonate/sodium carbonate and other buffer systems known in the art.

After removal of the residual liquids, the growth factor concentrate can be combined with other materials in a range of about 1 to about 20, about 1 to about 10, about 1 to about 1, and ranges therein. In some embodiments, the growth factor concentrate combined with other materials can be in the form of a paste or putty. In some embodiments, the growth factor concentrate can be slurried or dissolved in a solution and injected into an implant or coated over the surface of an implant. Agents to be combined with the growth factor concentrate to form a slurry or solution or other at least partially liquid mixture can include, but are not limited to, aqueous buffers, acetone, ethyl acetate, ethanol, isopropanol, and dimethyl sulfoxide (DMSO) and combinations thereof. Suitable buffers include, but are not limited to, Hank's balanced salt solution, phosphate buffered saline, phosphate solutions, saline or combinations thereof. The implant can include bone, connective tissue, tendon, pericardium, dermis, cornea, dura matter, fascia, heart valve, ligament, capsular graft, cartilage, collagen, nerve, placental tissue, and combinations thereof. The biological tissues can be allogeneic, autogeneic, or xenogeneic. In some embodiments when the implant is bone, the bone material can be cortical bone, cancellous bone or combination thereof. The bone can be mineralized, fully demineralized, partially demineralized, or a combination of the foregoing. DBM for use by the disclosed method can be prepared using any method or techniques known in the art, for a typical demineralization protocol, for example U.S. Pat. No. 5,314,476, or U.S. Pat. No. 8,574,825, each of which is incorporated in their entirety by reference. In further embodiments, the growth factor concentrate can be combined with other materials including antimicrobial agents. Examples include, but are not limited to, bisbiguanides, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillins, rifamycins, sulfonamides and tetracyclines. The foregoing antimicrobial agents may be used individually or as a mixture of multiple antimicrobial agents.

In other embodiments, an implant can be rolled into a solid form of the growth factor concentrate to “powder” the surface and produce a coated implant. Multiple layers of the coating can be applied to an implant. Methods of coating the surface of the implant include, but are not limited to, spraying, dip coating, pouring. The coating can be dried on the implant at a temperature between about 0° C. and about 50° C. The coating can be dried on the implant for times of less than a minute up to 24 hours, in some embodiments about 20 seconds, about 5 minutes, about 30 minutes, about 1 hour, about 2 hours, about 5 hours, about 10 hours, about 12 hours, about 15 hours, about 20 hours, about 24 hours, or within any range defined between two of the foregoing numbers, or a value or range within the largest range. The total thickness of the coating can be between about 0.1 mm and about 3 mm, in some embodiments about 0.1 mm, about 0.25 mm about 0.5 mm, about 1 mm, about 1.5 mm, about 2 mm, about 2.5 mm, about 3 mm, or within any range defined between two of the foregoing numbers, or a value or range within the largest range. When multiple layers are applied, each layer can be between about 0.1 mm and about 1 mm in thickness.

An aspect of the invention is a method of three-dimensional printing a growth factor concentrate-based implant. The method includes combining the growth factor concentrate and a binding agent within a three-dimensional printer and printing the implant of desired dimensions. The implant can be printed onto or into a separate medical implant or medical device. The implant can fully encapsulate, or partially cover the medical implant or medical device. The hybrid of the implant material and the medical implant or device can be used in other applications. For example, a hybrid implant can be implanted into a patient. Solvents can be combined with the biomaterial in the presence or absence of the binding agents to facilitate three-dimensional printing. The solvents can include, but are not limited to, water, alcohols, biocompatible organic solvents, buffers, or combinations thereof. Suitable biocompatible organic solvents include, but are not limited to, acetonitrile, dimethyl sulfoxide, acetone, ethyl acetate or combinations thereof. Suitable alcohols include, but are not limited to, ethanol, isopropanol, methanol or combinations thereof. Suitable buffers include, but are not limited to, Hank's balanced salt solution, phosphate buffered saline, saline or combinations thereof. Suitable binding agents include polymers, glues, gums, sugars, cellulose ethers, resins, and combinations thereof. The mass ratio of the growth factor concentrate to the solvent can be between about 10:1 and 0.01:1, about 1:1 to about 0.1 to 1, and ranges or values therein. The mass ratio of the growth factor concentrate to the solvent can be about 10:1, about 9:1, about 8:1, about 7:1, about 6:1, about 5:1, about 4:1, about 3:1, about 2:1, about 1:1, about 0.5:1, about 0.25:1, or about 0.01:1, or within any range defined between two of the foregoing numbers, or a value or range within the largest range. When a binder is used, the mass ratio of the binder to solvent can be between about 10:1 and about 0.01:1 to form a solution comprising the binder and solvent. In some embodiments, the mass ratio of the binder to solvent can be about 10:1, about 9:1, about 8:1, about 7:1, about 6:1, about 5:1, about 4:1, about 3:1, about 2:1, about 1:1, about 0.5:1, about 0.25:1, or about 0.01:1, or within any range defined between two of the foregoing numbers, or a value or range within the largest range. The mass ratio of the solution to growth factor concentrate can be between about 20:1, about 10:1 and about 1:1. In some embodiments, the mass ratio of the solution to the growth factor concentrate can be about 20:1, about 18:1, about 16:1, about 14:1, about 12:1, about 10:1, about 9:1, about 8:1, about 7:1, about 6:1, about 5:1, about 4:1, about 3:1, about 2:1, about 1:1, about 0.5:1, about 0.25:1, or about 0.01:1, or within any range defined between two of the foregoing numbers, or a value or range within the largest range.

In some embodiments, the growth factor concentrate may be combined with preserving agents to stabilize the growth factors during storage and implantation. The preserving agents may bind, stabilize, and enhance the activity and shelf life of growth factors. The mass ratio of the preserving agent to the growth factor concentrate can be between about 1:1 to about 1:100, about 1:10 to about 1:50. In some embodiments, the mass ratio of the preserving agent to the growth factor concentrate can be between about 1:1, about 1:2, about 1:5, about 1:10, about 1:25, about 1:50, about 1:75, about 1:100, or within any range defined between two of the foregoing numbers, or a value or range within the largest range. In some embodiments the growth factors may be combined with biodegradeable polymers. Polymers may be selected from, but are not limited to, the group consisting of alginates, carboxymethyl celluloses, hyaluronates, gelatins, glycerols, lecithins, phospholipids, polylactides, polyglycolides, polyethylene glycols, and copolymers, and combinations thereof. The mass ratio of the polymer to growth factor concentrate can be between about 1:1 and about 10:1. In some embodiments, the mass ratio of the polymer to growth factor concentrate can be about 1:1, about 2:1, about 3:1, about 5:1, about 7:1, about 10:1, or within any range defined between two of the foregoing numbers, or a value or range within the largest range.

An aspect of the invention is a growth factor concentrate. The growth factor concentrate can include at least one growth factor at a concentration between about 10,000 picograms of the growth factor per gram of an isolated concentrate and about 20,000 picograms of the growth factor per gram of an isolated concentrate and about 1000 picograms of the growth factor per gram of an isolated concentrate and about 100 picograms of the growth factor per gram of an isolated concentrate. In some embodiments, the growth factor concentrate can be about 100 picograms of the growth factor per gram of an isolated concentrate, about 1,000 picograms of the growth factor per gram of an isolated concentrate, about 2000 picograms of the growth factor per gram of an isolated concentrate, about 3000 picograms of the growth factor per gram of an isolated concentrate, about 4000 picograms of the growth factor per gram of an isolated concentrate, about 5000 picograms of the growth factor per gram of an isolated concentrate, about 6000 picograms of the growth factor per gram of an isolated concentrate, about 7000 picograms of the growth factor per gram of an isolated concentrate, about 8000 picograms of the growth factor per gram of an isolated concentrate, about 9000 picograms of the growth factor per gram of an isolated concentrate, about 10,000 picograms of the growth factor per gram of an isolated concentrate, about 12,000 picograms of the growth factor per gram of an isolated concentrate, about 14,000 picograms of the growth factor per gram of an isolated concentrate, about 16,000 picograms of the growth factor per gram of an isolated concentrate, about 18,000 picograms of the growth factor per gram of an isolated concentrate, or about 20,000 picograms of the growth factor per gram of an isolated concentrate, or any value or range between any two of these values.

An aspect of the invention is an implant comprising a growth factor concentrate. The growth factor concentrate can include at least one growth factor at a concentration between about 20,000 picograms of the growth factor per gram of an isolated concentrate and about 10,000 picograms of the growth factor per gram of an isolated concentrate and about 1000 picograms of the growth factor per gram of an isolated concentrate and about 100 picograms of the growth factor per gram of an isolated concentrate. The implant can include a coating layer on at least a portion of a surface of the implant. The thickness of the coating layer can be between about 100 μm and about 3 mm. In some embodiments, the thickness of the coating can be uniform (within about 10% thickness) or non-uniform.

Ranges have been discussed and used within the forgoing description. One skilled in the art would understand that any sub-range within the stated range would be suitable, as would any number within the broad range, without deviating from the invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A decellularized growth factor concentrate derived from bone marrow having between about 100 pg of at least one growth factor per gram of the decellularized concentrate and about 1000 pg of at least one growth factor per gram of the decellularized concentrate.

2. The concentrate of claim 1, wherein the bone marrow source is allogenic, autogenic, or xenogenic.

3. The concentrate of claim 1, wherein the at least one growth factor is native bone tissue growth factor proteins.

4. The concentrate of claim 1, wherein the at least one growth factors is selected from bone morphogenetic proteins, insulin-like growth factors, transforming growth factor beta, and fibroblast growth factors.

5. The concentrate of claim 1, wherein a residual moisture content of the concentrate is between about 0% and about 6%.

6. The concentrate of claim 1, wherein the growth factor concentrate is combined with at least one component.

7. The concentrate of claim 6, wherein the growth factor concentrate is coated on at least a portion of a surface of the at least one component.

8. The concentrate of claim 6, wherein a ratio of the growth factor concentrate to the at least one component is between 1:1 and 0.1:1.

9. The concentrate of claim 1, wherein the growth factor concentrate is used as an osteoinductive implant.

10. The concentrate of claim 6, wherein the at least one component is a carrier.

11. The concentrate of claim 11, wherein the carrier is a bone matrix.

12. A method of generating a decellularized growth factor concentrate comprising:
a. centrifuging bone marrow of the layered product to produce a purified layered product;
b. discarding red blood cells of the layered product to produce a purified layered product;
c. adding acid to the purified layered product to produce an acidified mixture;
d. mixing the acidified mixture to produce a second mixture;
e. settling the second mixture to produce a layered mixture;
f. removing the top layer of the mixture to produce a purified mixture;
g. adding base to the purified mixture to raise a pH of the purified mixture to produce a third mixture of a pH above 6; and
h. removing liquid from the third mixture to produce decellularized growth factor concentrate.

13. The method of claim 12, wherein the acid is at least one of hydrochloric acid, phosphoric acid, citric acid, or acetic acid.

14. The method of claim 12, wherein the removal of the remaining liquids occurs by at least method of decanting, filtration, or lyophilization.

15. The method of claim 12, further comprising filtering the bone marrow prior to step a.

16. The method of claim 12, further comprising reducing a size of the bone marrow to less than about 1 mm in size prior to step a.

17. The method of claim 12, wherein the base is at least one of hydroxide, phosphate, carbonate, citrate, silicate, and acetate.
